(19) United States
(12) Patent Application Publication
(10) Pub. No.: US 2007/0178440 A1
(43) Pub. Date: Aug. 2, 2007

(54) Inhibitors of gob-4 protein as asthma therapeutics

(75) Inventor: Maximillian T. Follettie, Belmont, MA (US)

Correspondence Address: WilmerHale/Wyeth, 60 STATE STREET, BOSTON, MA, 02109, US

(73) Assignee: wyeth, Madison, NJ (US)

(21) Appl. No.: 10/591,225

(22) PCT Filed: Mar. 18, 2005
(86) PCT No.: PCT/US05/08946

§ 371 Date: Apr. 3, 2007

Related U.S. Application Data
(60) Provisional application No. 60/554,765, filed on Mar. 19, 2004.

(51) Int. Cl. G01N 33/53; C12Q 1/00

(52) U.S. Cl. 435/4; 435/7.1

(57) ABSTRACT

Methods of screening for agents for treating asthma are provided. The methods involve screening for agents that decrease the production or activity of a Gob-4 protein that has been discovered herein to play a role in producing the symptoms and pathological complications involved in asthma. Methods of treating asthma, as well as screening for and treating with inhibitors of a Gob-4 protein are also provided.

### FIELD OF THE INVENTION

It is an object of the invention to provide methods of screening for agents for treating asthma. It is a further object of the invention to provide methods for treating asthma. These and other objects and advantages of the present invention will be apparent from the descriptions herein.

### BACKGROUND OF THE INVENTION

The present invention relates generally to asthma therapeutics. Specifically, the invention relates to methods of screening for agents for treating asthma and methods for treating asthma.

Asthma is a chronic inflammatory disease of the airways characterized by recurrent episodes of reversible airway obstruction and airway hyperresponsiveness (AHR). Typical clinical manifestations include shortness of breath, wheezing, coughing and chest tightness that can become life threatening or fatal. While existing therapies focus on reducing the symptomatic bronchospasm and pulmonary inflammation, there is a growing awareness of the role of long term airway remodeling in accelerated lung deterioration in asthmatics. Airway remodeling refers to a number of pathological features including epithelial smooth muscle and myofibroblast hyperplasia and/or metaplasia, subepithelial fibrosis and matrix deposition. The processes collectively result in up to about 300% thickening of the airway in cases of fatal asthma. Despite the considerable progress that has been made in elucidating the pathophysiology of asthma, the prevalence, morbidity, and mortality of the disease has increased during the past two decades. In 1995, in the United States alone, nearly 1.8 million emergency room visits, 466 thousand hospitalizations and 5,429 deaths were directly attributed to asthma.

It is generally accepted that allergic asthma is initiated by an inappropriate inflammatory reaction to airborne allergens. The lungs of asthmatics demonstrate an intense infiltration of lymphocytes, mast cells and especially eosinophils. A large body of evidence has demonstrated that this immune response is driven by CD4⁺ T-cells expressing a T_{H}2 cytokine profile. One murine model of asthma involves sensitization of the animal to ovalbumin (OVA) followed by intratracheal delivery of the OVA challenge. This procedure generates a T_{H}2 immune reaction in the mouse lung and mimics four major pathophysiological responses seen in human asthma, including upregulated serum IgE (atopy), eosinophilia, excessive mucus secretion, and AHR. The cytokine IL-13, expressed by basophils, mast cells, activated T cells and NK cells, plays a central role in the inflammatory response to OVA in mouse lungs. Direct lung instillation of murine IL-13 elicits all four of the asthma-related pathologies and, conversely, the presence of a soluble IL-13 antagonist (sIL-13Rα2-Fc) completely blocked both the OVA-challenge induced goblet cell mucus synthesis and the AHR to acetylcholine. Wills-Karp, M., et al., “Interleukin-13: central mediator of allergic asthma,” Science 282(5397): 2258-2261 (1998); Grunig, G., et al., “Requirement for IL-13 independently of IL-4 in experimental asthma,” Science 282(5397): 2261-2263 (1998). Thus, IL-13 mediated signaling is sufficient to elicit all four asthma-related pathophysiological phenotypes and is required for the hypersecretion of mucus and induced AHR in the mouse model.

Biologically active IL-13 binds specifically to a low-affinity binding chain IL-13Rα1 and to a high-affinity multimeric complex composed of IL-13Rα1 and IL-4R, a shared component of IL-4 signaling complex. Wills-Karp, M., “IL-12/IL-13 axis in allergic asthma,” J Allergy Clin Immunol 107(1): 9-18 (2001). Activation of the IL-13 pathway cascade triggers the recruitment, phosphorylation and ultimate nuclear translocation of the transcriptional activator Stat6. A number of physiological studies demonstrate the inability of pulmonary OVA-challenge to elicit major pathology related phenotypes including eosinophil infiltration, mucus hypersecretion and airway hyperreactivity in mice homozygous for the Stat6^{-/-} null allele. Kuperman, D., et al., “Signal transducer and activator of transcription factor 6 (Stat6)-deficient mice are protected from antigen-induced airway hyperresponsiveness and mucus production,” J Exp Med 187(6): 939-48 (1998). Recent genetic studies have demonstrated a linkage between specific human alleles of IL-13 and its signaling components with asthma and atopy, demonstrating the critical role of this pathway in the human disease. Shirakawa et al., “Atopy and asthma: genetic variants of IL-4 and IL-13 signaling,” Immunol. Today 21(2):60-64 (2000).

IL-13 also binds to an additional receptor chain, IL-13Rα2, expressed in both human and mouse with as yet undefined biological function. The murine IL-13Rα2 binds IL-13 with approximately 100-fold greater affinity (Kd of 0.5 to 1.2 nM) relative to IL-13Rα1, allowing the construction of a potent soluble IL-13 antagonist, sIL-13Rα2-Fc. The sIL-13Rα2-Fc has been used as an antagonist in a variety of disease models to demonstrate the role of IL-13 in Schistosomiasis induced liver fibrosis and granuloma formation, tumor immune surveillance, as well as in the OVA-challenge asthma model. Wills-Karp, M., et al., “Interleukin-13: central mediator of allergic asthma,” Science 282(5397): 2258-2261 (1998); Grunig, G., et al., “Requirement for IL-13 independently of IL-4 in experimental asthma,” Science 282(5397): 2261-2263 (1998).

Gob-4 was originally cloned from mouse intestinal epithelial cells and is expressed at known cellular sites of mucin expression including stomach mucous-neck cells and intestinal goblet cells. The Xenopus ortholog of human Gob-4, designated XAG-2, is expressed in the cement gland, a secretory organ that expresses a mucin-like polymer allowing the Xenopus embryo to attach to solid supports. XAG-2 is a secreted molecule with anterior ectoderm patterning activity capable of inducing ectopic cement gland formation. This data supports the concept that human Gob-4 plays a key role in the mucus-secreting goblet cell hyperplasia that is the hallmark of asthma. Aliases for GOB-4 are hAG-2 and AGR-2.

Current therapy of asthma to treat bronchospasms and airway inflammation includes use of bronchodilators, corticosteroids, and leukotriene inhibitors. Many of such treatments include undesired side effects and lose effectiveness after being used for a period of time. Additionally, limited agents for therapeutic intervention are available that decrease the airway remodeling process that occurs in asthmatics. Therefore, there remains a need for an increased molecular understanding of asthma, coupled to identification of novel therapeutic strategies to combat this complex disease. The present invention addresses these needs.

### SUMMARY OF THE INVENTION

It has been discovered that the messenger RNA (mRNA) of Gob-4 protein is statistically significantly increased in an animal model of asthma compared to control, non-asthmatic animals. Specifically, the mRNA encoding Gob-4 protein has been found to be elevated by either intratracheal ovalbumin challenge or direct pulmonary instillation of IL-13 and has herein been discovered as a target for asthma therapeutics. Accordingly, in one aspect of the invention, methods of screening for agents for treating asthma are provided. Methods for treating asthma are also provided.

In one aspect of the invention, a method of screening for agents for treating asthma includes (a) contacting a Gob-4 protein with a test agent thought to be effective in inhibiting the activity of the Gob-4 protein; (b) determining if the test agent inhibits the activity of the Gob-4 protein; and (c) classifying the test agent as an agent for treating asthma if the test agent inhibits the activity of the Gob-4 protein.

In another aspect, the invention provides a method of screening for agents for treating asthma by (a) contacting a nucleotide sequence encoding a reporter gene product operably linked to a Gob-4 protein promoter with a test agent thought to be effective in inhibiting production of a Gob-4 protein; (b) determining if the test agent inhibits production of the reporter gene product; and (c) classifying the test agent as an agent for treating asthma if the test agent inhibits production of the reporter gene product.

In yet another aspect of the invention, methods for treating asthma are provided. In one embodiment, a method includes administering to a mammal in need thereof a therapeutic amount of an agent that decreases the activity of a Gob-4 protein. In a further embodiment of the invention, a method includes administering to a mammal in need thereof a therapeutic amount of an agent that decreases the production of a Gob-4 protein.

### DESCRIPTION OF THE FIGURES

FIG. 1 is a representation of the nucleotide (cDNA) sequence of human Gob-4 protein (NCBI nucleotide database, accession no. NM_{—}006408) (SEQ ID NO:1).

FIG. 2 is a representation of the amino acid sequence of human Gob-4 protein (NCBI protein database, accession no. AAP97179) (SEQ ID NO:2).

FIG. 3 is a representation of the nucleotide (cDNA) sequence of mouse Gob-4 protein as disclosed by Komiya T., et al., “Cloning of the gene Gob-4, which is expressed in intestinal goblet cells in mice,” Biochim Biophys Acta, Mar. 19, 1999; 1444(3): 434-438 (SEQ ID NO:3). The coding sequence begins at residue 53 and ends at residue 580.

FIG. 4 is a representation of the amino acid sequence for mouse Gob-4 protein as disclosed by Komiya T., et al. (SEQ ID NO:4).

FIG. 5 is a representation of the nucleotide (cDNA) sequence of rat Gob-4 protein as disclosed on the NCBI nucleotide database, identified by sequence homology (accession no. XM 216691.2) (SEQ ID NO:5). The coding sequence begins at residue 54 and ends at residue 581.

FIG. 6 is a representation of the amino acid sequence for rat Gob-4 protein (accession no. XM 216691.2) (SEQ ID NO:6).

FIG. 7 (SEQ ID NO:7) is a representation of the nucleotide sequence of the promoter for human Gob-4 protein.

FIG. 8 (SEQ ID NO:8) is a representation of the nucleotide sequence of the promoter for human Gob-4 protein.

### DETAILED DESCRIPTION OF THE INVENTION

The patent and scientific literature referred to herein establishes knowledge that is available to those of skill in the art. The issued U.S. patents, allowed applications, published applications (U.S. and foreign) and references, including GenBank database sequences, that are cited herein are incorporated by reference to the same extent as if each was specifically and individually indicated to be incorporated by reference.

The invention is based upon the unexpected discovery that the mRNA of Gob-4 protein is significantly increased in an animal model of asthma compared to control, non-asthmatic animals. Specifically, the mRNA encoding Gob-4 protein is elevated by either intratracheal ovalbumin challenge or direct pulmonary instillation of IL-13. Gob-4 was originally cloned from mouse intestinal epithelial cells and is expressed at known cellular sites of mucin expression. XAG-2, the Xenopus ortholog of mAGR2/mGob-4, is expressed in the cement gland, a secretory organ that expresses a mucin-like polymer that allows the Xenopus embryo to attach to solid supports. XAG-2 is capable of inducing ectopic cement gland formation. Goblet cell metaplasia and/or hyperplasia is observed in airway epithelium following pulmonary allergic challenge. Gob-4 is considered herein to be involved in the goblet cell metaplasia and/or hyperplasia and resultant mucus hyperproduction observed in allergic asthma. Specifically, the inventors believe that Gob-4 acts as a developmental switch to help epithelial cells differentiate into goblet cells. Due to the involvement of Gob-4 proteins in the allergic response in asthma, an inhibitor of the Gob-4 proteins will be effective in treating asthma. Thus, the inventors believe that Gob-4 protein is involved in the allergic response in asthma, and, consequently, that an inhibitor of the Gob-4 protein will be effective in treating asthma. Consequently, an inhibitor of Gob-4 protein should be effective to treat asthma. “Asthma”, as used herein includes, but is not limited to, atopic asthma, nonatopic asthma, allergic asthma, exercise-induced asthma, drug-induced asthma, occupational asthma and late stage asthma.

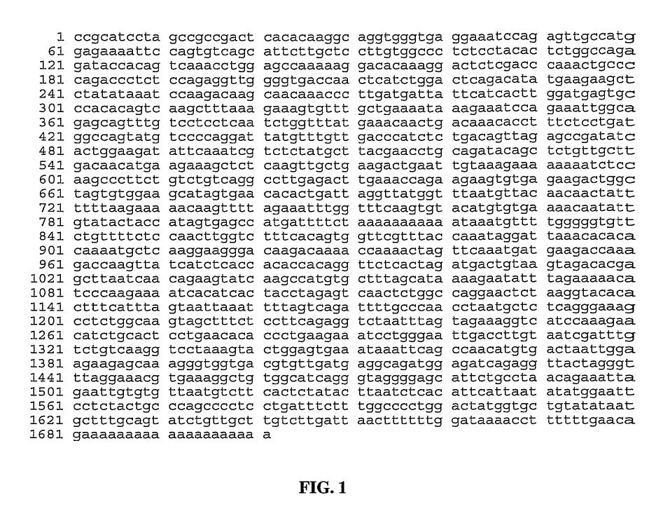

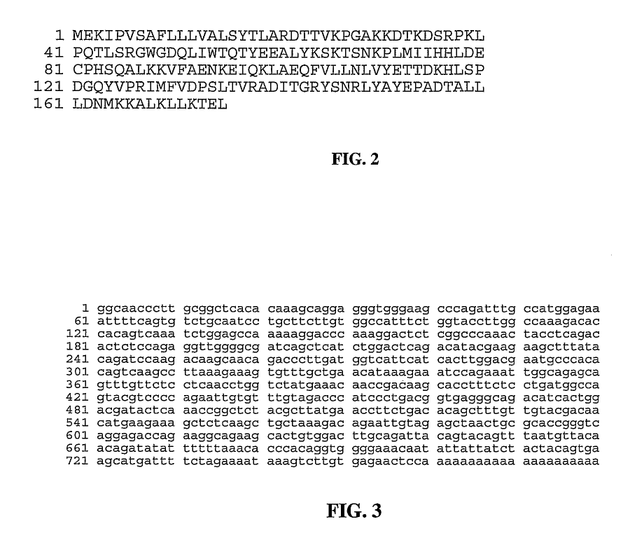

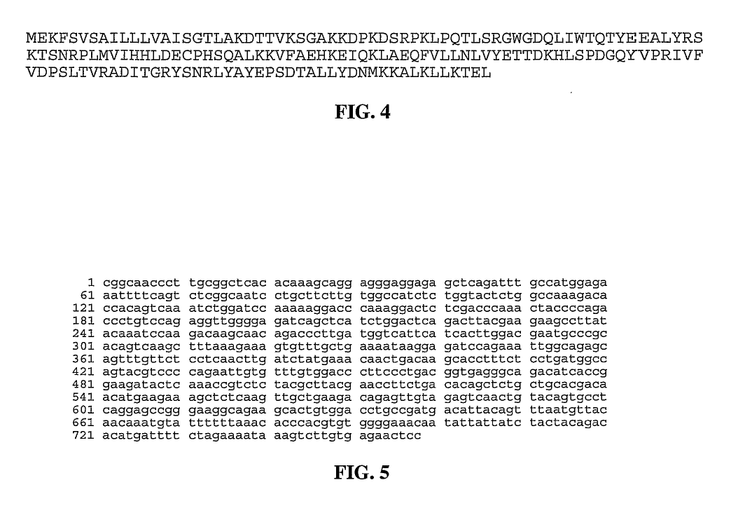

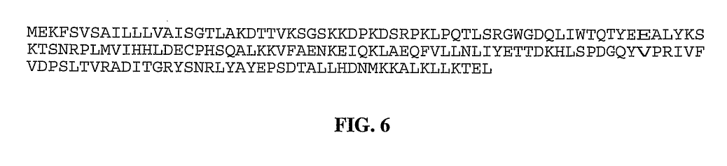

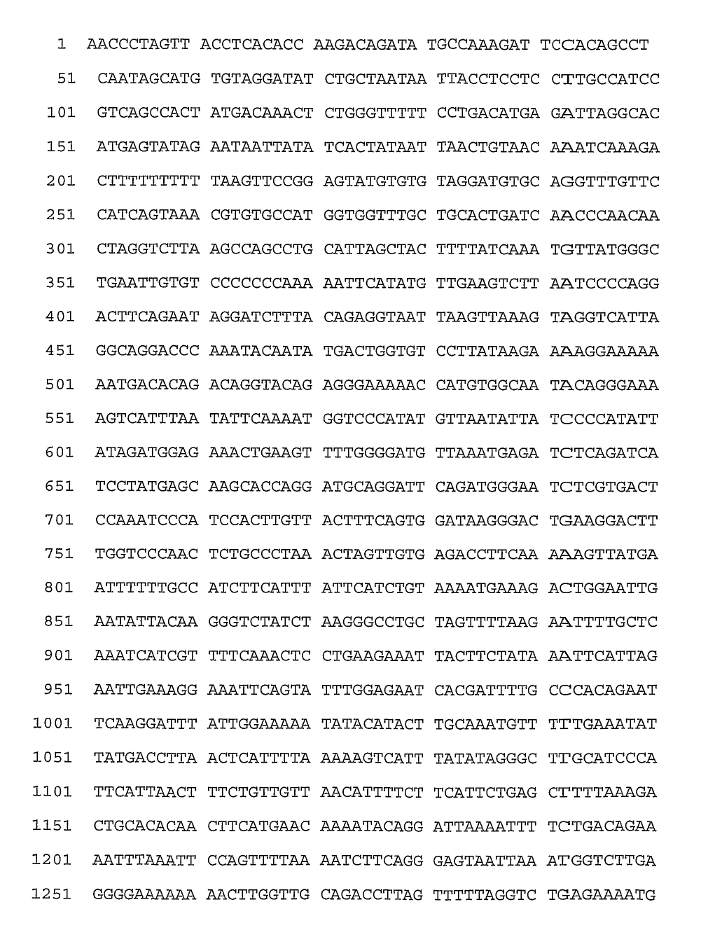

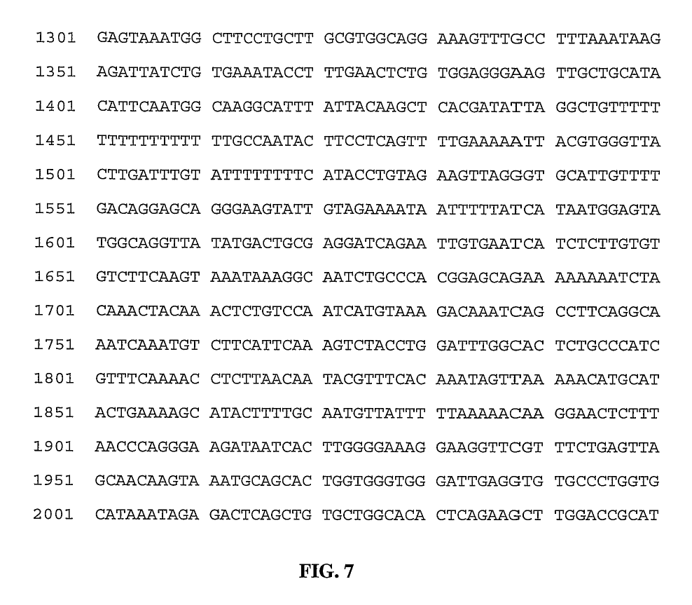

As noted above, the invention provides methods of screening for agents for treating asthma in a mammal. In one embodiment, the mammal is a human. As used herein, “agent” includes, but is not limited to, synthetic small molecules, chemicals, nucleic acids, peptides and proteins such as hormones, antibodies and portions thereof. In one aspect, the methods include contacting a Gob-4 protein with a test agent that is thought to be effective in modulating (e.g., inhibiting or increasing) the activity of Gob-4 protein. A “test agent” is a putative “agent,” the modulating ability of which has not yet been confirmed. Once test agents are screened, they are classified as “agents,” if they are shown to modulate protein activity or transcription. In a particular embodiment, the activity of Gob-4 protein is inhibited. The methods include: determining if the test agent modulates (e.g., inhibits) the activity of the Gob-4 protein and classifying the test agent as an agent for treating asthma if the test agent modulates (e.g., inhibits) the activity of the Gob-4 protein. The nucleotide and amino acid sequences of human Gob-4 protein are set forth in SEQ ID NO:1 and SEQ ID NO:2, as provided in FIGS. 1 and 2, respectively. The nucleotide and amino acid sequences of murine Gob-4 protein are set forth in SEQ ID NO:3 and SEQ ID NO:4, as provided in FIGS. 3 and 4, respectively. The nucleotide and amino acid sequences of rat Gob-4 protein are set forth in SEQ ID NO:5 and SEQ ID NO:6, as provided in FIGS. 5 and 6, respectively.

The discovery that Gob-4 protein is associated with inducing the symptoms and/or complications of asthma renders the sequences of Gob-4 protein useful in methods of identifying agents of the invention. Such methods include assaying potential agents for the ability to inhibit Gob-4 protein activity. Polynucleotides and polypeptides useful in these assays include not only the genes and encoded polypeptides disclosed herein, but also variants thereof that have substantially the same activity as wild-type genes and polypeptides. “Variants” as used herein, includes polynucleotides or polypeptides containing one or more deletions, insertions or substitutions, as long as the variant retains substantially the same activity of the wild-type polynucleotide or polypeptide. With regard to polypeptides, deletion variants are contemplated to include fragments lacking portions of the polypeptide not essential for biological activity, and insertion variants are contemplated to include fusion polypeptides in which the wild-type polypeptide or fragment thereof has been fused to another polypeptide.

Accordingly, the Gob-4 protein utilized in the invention may be encoded by a nucleotide sequence that has at least about 60%, at least about 70%, at least about 80% or at least about 90% identity to the nucleotide sequence set forth in SEQ ID NO:1 (FIG. 1), SEQ ID NO:3 (FIG. 3), or SEQ ID NO:5 (FIG. 5). Percent identity may be determined, for example, by comparing sequence information using the advanced BLAST computer program, version 2.0.8, available from the National Institutes of Health.

Additionally, the Gob-4 protein may be encoded by nucleotide sequences having substantial similarity to the nucleotide sequence set forth in SEQ ID NO:1 (FIG. 1), SEQ ID NO:3 (FIG. 3), or SEQ ID NO:5 (FIG. 5). “Substantial similarity,” as used herein means that the nucleotide sequence is sufficiently similar to a reference nucleotide sequence that it will hybridize therewith under moderately stringent conditions. This method of determining similarity is well known in the art to which the invention pertains. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| | | | Hybridization | |
|---|---|---|---|---|
| Stringency | Polynucleotide | Hybrid Length | Temperature and | Wash Temperature |
| Condition | Hybrid | (bp)¹ | Buffer² | and Buffer² |
| A | DNA:DNA | >50 | 65° C.; 1 × SSC -or- | 65° C.; 0.3 × SSC |
| | | | 42° C.; 1 × SSC, | |
| | | | 50% formamide | |
| B | DNA:DNA | <50 | T_{B}*; 1 × SSC | T_{B}*; 1 × SSC |
| C | DNA:RNA | >50 | 67° C.; 1 × SSC -or- | 67° C.; 0.3 × SSC |
| | | | 45° C.; 1 × SSC, | |
| | | | 50% formamide | |
| D | DNA:RNA | <50 | T_{D}*; 1 × SSC | T_{D}*; 1 × SSC |
| E | RNA:RNA | >50 | 70° C.; 1 × SSC -or- | 70° C.; 0.3 × SSC |
| | | | 50° C.; 1 × SSC, | |
| | | | 50% formamide | |
| F | RNA:RNA | <50 | T_{F}*; 1 × SSC | T_{f}*; 1 × SSC |
| G | DNA:DNA | >50 | 65° C.; 4 × SSC -or- | 65° C.; 1 × SSC |
| | | | 42° C.; 4 × SSC, | |
| | | | 50% formamide | |
| H | DNA:DNA | <50 | T_{H}*; 4 × SSC | T_{H}*; 4 × SSC |
| I | DNA:RNA | >50 | 67° C.; 4 × SSC -or- | 67° C.; 1 × SSC |
| | | | 45° C.; 4 × SSC, | |
| | | | 50% formamide | |
| J | DNA:RNA | <50 | T_{J}*; 4 × SSC | T_{J}*; 4 × SSC |
| K | RNA:RNA | >50 | 70° C.; 4 × SSC -or- | 67° C.; 1 × SSC |
| | | | 50° C.; 4 × SSC, | |
| | | | 50% formamide | |
| L | RNA:RNA | <50 | T_{L}*; 2 × SSC | T_{L}*; 2 × SSC |
| M | DNA:DNA | >50 | 50° C.; 4 × SSC -or- | 50° C.; 2 × SSC |
| | | | 40° C.; 6 × SSC, | |
| | | | 50% formamide | |
| N | DNA:DNA | <50 | T_{N}*; 6 × SSC | T_{N}*; 6 × SSC |
| O | DNA:RNA | >50 | 55° C.; 4 × SSC -or- | 55° C.; 2 × SSC |
| | | | 42° C.; 6 × SSC, | |
| | | | 50% formamide | |
| P | DNA:RNA | <50 | T_{P}*; 6 × SSC | T_{P}*; 6 × SSC |
| Q | RNA:RNA | >50 | 60° C.; 4 × SSC -or- | 60° C.; 2 × SSC |
| | | | 45° C.; 6 × SSC, | |
| | | | 50% formamide | |
| R | RNA:RNA | <50 | T_{R}*; 4 × SSC | T_{R}*; 4 × SSC |
| ¹The hybrid length is that anticipated for the hybridized region(s) of | | | | |
| # the hybridizing polynucleotides. When hybridizing a polynucleotide to a target polynucleotide | | | | |
| # of unknown sequence, the hybrid length is assumed to be that of the hybridizing | | | | |
| # polynucleotide. When polynucleotides of known sequence are hybridized, the hybrid length | | | | |
| # can be determined by aligning the sequences of the polynucleotides and identifying the | | | | |
| # region or regions of optimal sequence complementarity. | | | | |
| ²SSPE (1 × SSPE is 0.15M NaCl, 10 mM NaH₂PO₄, and 1.25 mM EDTA, pH | | | | |
| # 7.4) can be substituted for SSC (1 × SSC is 0.15M NaCl and 15 mM sodium citrate) in the | | | | |
| # hybridization and wash buffers; washes are performed for 15 minutes after hybridization is complete. | | | | |
| T_{B}*-T_{R}*: The hybridization temperature for hybrids anticipated to be | | | | |
| # less than 50 base pairs in length should be 5-10EC less than the melting | | | | |
| # temperature (Tₘ) of the hybrid, where Tₘ is determined according to the following | | | | |
| # equations. For hybrids less than 18 base pairs in | | | | |
| # length, Tₘ(EC) = 2(# of A + T bases) + 4(# of G + C bases). For | | | | |
| # hybrids between 18 and 49 base pairs in | | | | |
| # length, Tₘ(EC) = 81.5 + 16.6(log₁₀Na+) + 0.41(% G + C) − (600/N), where | | | | |
| # N is the number of bases in the hybrid, and Na+ is the concentration of sodium ions in | | | | |
| # the hybridization buffer (Na+ for 1 × SSC = 0.165 M). | | | | |

Additional examples of stringency conditions for polynucleotide hybridization are provided in Sambrook et al., “Molecular Cloning: A Laboratory Manual”, Chs. 9 & 11, Cold Spring Harbor Laboratory Press, Cold Spring Harbor, N.Y. (1989), and Ausubel et al., eds., Current Protocols in Molecular Biology, §§ 2.10, 6.3-6.4, John Wiley & Sons, Inc. (1995), herein incorporated by reference.

Gob-4 protein may be produced by methods known to the skilled artisan. For example, a nucleotide sequence encoding a Gob-4 protein gene may be introduced into a desired host cell. Such a nucleotide sequence may first be inserted into an appropriate recombinant expression vector.

Recombinant expression vectors may be constructed by incorporating the above-recited nucleotide sequences within a vector according to methods well known to the skilled artisan. A wide variety of vectors are known that are useful in the invention. Suitable vectors include plasmid vectors and viral vectors, including retrovirus vectors, adenovirus vectors, adeno-associated virus vectors and herpes viral vectors. The vectors may include other known genetic elements necessary or desirable for efficient expression of the nucleic acid in a specified host cell, including regulatory elements. For example, the vectors may include a promoter and any necessary enhancer sequences that cooperate with the promoter to achieve transcription of the gene. The nucleotide sequence may be operably linked to such regulatory elements.

As used herein, a nucleotide sequence is “operably linked” to another nucleotide sequence when it is placed in a functional relationship with another nucleotide sequence. For example, if a coding sequence is operably linked to a promoter sequence, this generally means that the promoter may promote transcription of the coding sequence. Operably linked means that the DNA sequences being linked are typically contiguous and, where necessary to join two protein coding regions, contiguous and in reading frame. However, since enhancers may function when separated from the promoter by several kilobases and intron sequences may be of variable lengths, some nucleotide sequences may be operably linked but not contiguous. A wide variety of methods are available for introducing the nucleotide sequence encoding a Gob-4 protein, and which may be included in a recombinant expression vector, into a host cell. Such methods are known to the art and include mechanical methods, chemical methods, lipophilic methods and electroporation. Microinjection and use of a gene gun with, for example, a gold particle substrate for the DNA to be introduced is a representative, non-limiting exemplary mechanical method. Use of calcium phosphate or DEAE-Dextran is a representative, non-limiting exemplary chemical method. Exemplary lipophilic methods include use of liposomes and other cationic agents for lipid-mediated transfection. Such methods are well known to the art.

A wide variety of host cells may be utilized in the present invention to produce the desired quantities of a Gob-4 protein. Such cells include, but are not limited to, eukaryotic and prokaryotic cells, including mammalian cells and bacterial cells known to the art.

The Gob-4 protein may be isolated and purified by techniques well known to the skilled artisan, including, but not limited to, chromatographic, electrophoretic and centrifugation techniques. Such methods are known to the art.

The Gob-4 protein is typically contacted with a test agent for a time period sufficient to inhibit the activity of the Gob-4 protein. This time period may vary depending on the nature of the inhibitor and the Gob-4 protein selected. The skilled artisan without undue experimentation may readily determine such times. An exemplary test agent is one that binds to or otherwise decreases the activity of the Gob-4 protein, although test agents that inhibit Gob-4 protein by, for example, binding to Gob-4 protein blocking interaction with receptors or proteins involved in signal transduction processes, or by some other mechanism, are also envisioned.

A wide variety of assays may be utilized to determine whether the test agent inhibits the activity of the Gob-4 protein. As the Gob-4 proteins are involved in the differentiation of epithelial cells into goblet cells, one assay for the activity of Gob-4 is quantitation of mucus produced from the goblet cells stained with H&E, a technique known to those of skill in the art. In one embodiment, epithelial cells are contacted or otherwise incubated with an effective amount of a Gob-4 protein and the test agent. This amount is effective for stimulating differentiation of the epithelial cell cells into goblet cells and can be determined by the skilled artisan. Mucus production may then be measured and may be compared to control cells treated with the Gob-4 protein in the absence of the test agent. In addition to H&E staining for mucus production, decreases in mucus encoding mRNA expression can be measured using classical reverse transcriptase polymerase chain reaction (RT-PCR), global profiling of expressed mRNA using cDNA or oligonucleotide arrays, or quantitating the amount of mucopolysaccharides in the mucus.

A wide variety of assays are available for quantitating mucus, including use of stains to stain mucosal components and quantitation by a colorimetric assay. In one embodiment, a periodic acid Schiff technique may be used to stain mucins, glycoproteins or mucopolysaccharides present in mucus. Alternatively, mucins may be radiolabelled and analyzed by hydrophobic interaction chromatography as described in Svitacheva, N. and Davies, J. R., “Mucin biosynthesis and secretion in tracheal epithelial cells in primary culture,” Biochem. J. 353: 23-32 (2001). The mucus may be isolated by standard methods known to the art and as described, for example, in Svitacheva, N. and Davies, J. R., supra. Briefly, cells in culture may be harvested and the mucins may be isolated by isopycnic density-gradient centrifugation after dialyzing the samples.

Mammalian epithelial cells from a wide variety of sources may be utilized in the above-referenced screening assays involving quantitation of mucin. Nonlimiting examples of epithelial cells are epithelial cells are small airway epithelial cells or bronchial epithelial cells, which can be obtained from Clonetics (www.cambrex.com). Such cells are typically present as a cell culture. Cell culture methods are known to the skilled artisan, and described in, for example, Ausubel et al., eds., Current Protocols in Molecular Biology, John Wiley & Sons; A. J. Shaw, ed., Epithelial Cell Culture: A Practice Approach (Practical Approach Series), IRL press, 1996; R. I. Freshney, and M. G. Freshney, eds., Culture of Epithelial Cells, 2nd Edition, John Wiley & Sons, 2002; and C. Wise, ed., Epithelial Cell Culture Protocols (Methods in Molecular Biology, v. 188), Humana Press, 2002. In addition, epithelial cell lines may be used in screening assays involving the quantitation of mucin. Alternatively, in yet another embodiment, the amount of epithelial cell-derived goblet cells may be quantitated. That is, the number of goblet cells formed from differentiation of epithelial cells by the action of Gob-4 may be quantitated. Methods of quantitation include, without limitation, staining for mucus production using periodic acid-Schiff (PAS)/acain blue and microscopic examination for increased numbers of goblet epithelial cells or an increase in secretory granuoles within specific cells. Kondo et al., “Interleukin-13 Induces Goblet Cell Differentiation in Primary Cell Culture from Guinea Pig Tracheal Epithelium” Am. J. Respir. Cell Mol. Biol. November 2002 27(5): 536-541.

A wide variety of test agents may be tested in the screening methods of the present invention. For example, small molecule compounds, known in the art, chemicals, nucleic acids, peptides and proteins such as hormones, antibodies, and portions thereof, may act as test agents. In one nonlimiting example, the three-dimensional structure of the active site of Gob-4 protein is determined by crystallizing the complex formed by the protein and a known inhibitor. Rational drug design is then be used to identify new test agents by making alterations in the structure of a known inhibitor or by designing small molecule compounds that bind to the active site of the protein.

The invention also provides a method of screening for agents for treating asthma in a mammal. In one embodiment, the invention includes contacting a nucleotide sequence encoding a reporter gene product operably linked to a Gob-4 protein promoter, with a test agent thought to be effective in inhibiting production of Gob-4 protein; determining if the test agent inhibits production of the reporter gene product; and classifying the test agent as an agent for treating asthma if the test agent inhibits production of the reporter gene product. In one embodiment, the mammal is a human. “Asthma”, as used herein includes, but is not limited to, atopic asthma, nonatopic asthma, allergic asthma, exercise-induced asthma, drug-induced asthma, occupational asthma and late stage asthma.

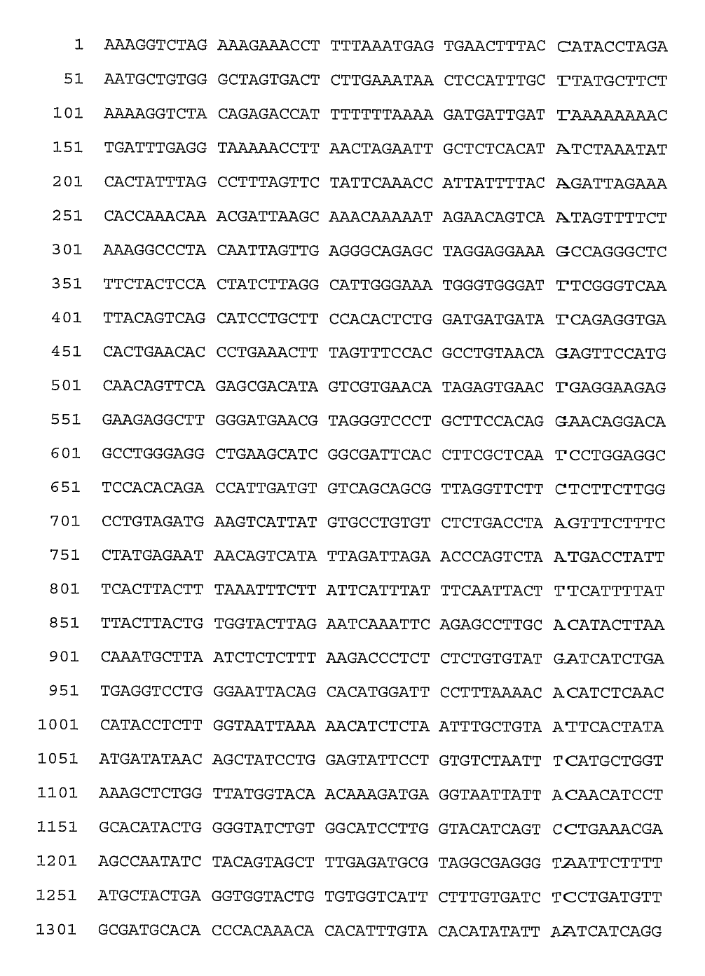

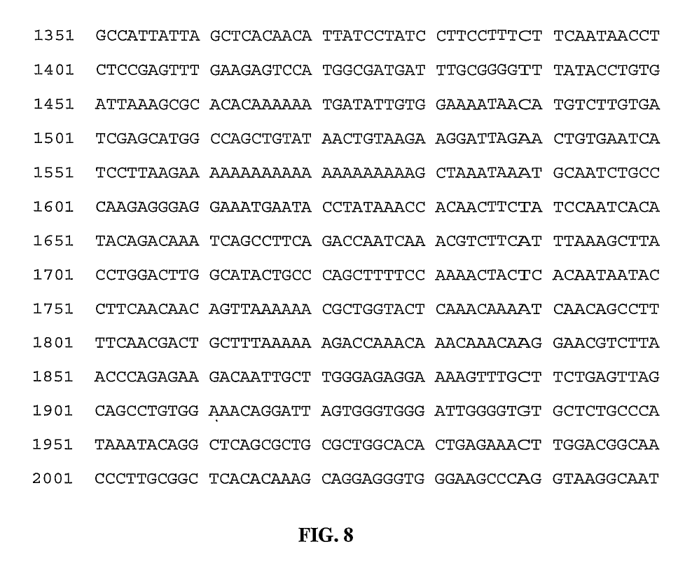

The Gob-4 protein promoter preferably includes a nucleotide sequence set forth in SEQ ID NO:7 or SEQ ID NO: 8, as set forth in FIGS. 7 and 8, respectively. SEQ ID NO:7 and SEQ ID NO:8 each depict the sequence located two kilobases upstream of the mRNA initiation codon. These sequences were identified electronically using publicly available sequence data known to those of skill in the art. Nucleotide sequences having at least about 50%, at least about 70%, at least about 80% and at least about 90% identity to such sequences and that function as promoter, for example, to direct expression of a gene encoding a Gob-4 protein described herein, are also encompassed in the invention.

The nucleotide sequence of the Gob-4 protein promoter is determined by art-recognized methods. One nonlimiting example of such a method is to screen a genomic library (e.g., a YAC human genomic library) for the promoter sequence of interest using SEQ ID NO:1 (FIG. 1), SEQ ID NO:3 (FIG. 3), or SEQ ID NO:5 (FIG. 5) as a probe. Another nonlimiting example of a method to determine the appropriate promoter sequence is to perform a Southern blot of the human genomic DNA by probing electrophoretically resolved human genomic DNA with a probe (e.g., a probe comprising SEQ ID NO:1 or a portion thereof) and then determining where the cDNA probe (e.g., SEQ ID NO:1) hybridizes. Upon determining the band to which the probe (e.g., SEQ ID NO:1) hybridizes, the band can be isolated (e.g., cut out of the gel) and subjected to sequence analysis. This allows detection of the nucleotide fragment 5′ of nucleotides 104-106 (i.e., the ATG site) of SEQ ID NO:1. The nucleotide fragment may be between approximately 500 to 1000 units in length. The promoter sequence for murine Gob-4 protein set forth in SEQ ID NO:3 (FIG. 3) or the promoter sequence for rat Gob-4 protein set forth in SEQ ID NO:5 (FIG. 5) may be determined by these methods as well. Nucleotide sequences having at least about 70%, at least about 80% and at least about 90% identity to such sequences and that function as promoter, for example, to direct expression of a gene encoding a Gob-4 protein described herein, are also encompassed in the invention.

A wide variety of reporter genes may be operably linked to the Gob-4 protein promoter described above. Such genes may encode, for example, luciferase, β-galactosidase, chloramphenical acetyltransferase, β-glucuronidase, alkaline phosphatase, and green fluorescent protein, or other reporter gene products known to the art.

In an embodiment of the invention, the nucleotide sequence encoding a reporter gene that is operably linked to a Gob-4 protein promoter is introduced into a host cell. Such a nucleotide sequence may first be inserted into an appropriate recombinant expression vector as previously described herein.

The vectors in this aspect of the invention may include other known genetic elements necessary or desirable for efficient expression of the nucleic acid sequence from the Gob-4 protein promoter in a specified mammalian cell, including regulatory elements. For example, the vectors may include any necessary enhancer sequences that cooperate with the promoter in vivo, for example, to achieve in vivo transcription of the reporter gene. The methods of introducing the nucleotide sequence into a host cell are identical to that previously described for producing the Gob-4 protein.

A wide variety of host cells may be utilized in the methods of screening in the present invention. Exemplary host cells include, for example, Chinese hamster ovary, E. coli, COS and Bacillus.

Alternatively, the nucleotide sequence encoding all or a portion of the Gob-4 protein gene may be utilized in the vector for the screening methods described herein. In such a case, Gob-4 protein may be isolated and purified by techniques well known to the skilled artisan, including chromatographic, electrophoretic and centrifugation techniques, as previously described herein. Additionally, Gob-4 protein may be quantified by methods known to the art.

After contacting a nucleotide sequence encoding a reporter gene, or a Gob-4 protein gene, operably linked to a Gob-4 protein promoter with a test agent thought to be effective in inhibiting production of a Gob-4 protein, it is determined if the test agent inhibits production of the reporter gene product. This endpoint may be determined by quantifying either the amount or activity of the reporter gene product. The method of quantification will depend on the reporter gene that is used, but may involve use of an enzyme-linked immunosorbent assay with antibodies to the reporter gene product. Additionally, the assay may measure chemiluminescence, fluorescence or radioactive decay, or other methods known in the art. Assays for determining the activity or amount of the reporter gene products described herein are known to the art. If the test agent inhibits production of the reporter gene product, it is classified as an agent for treating asthma.

The invention also provides methods for treating asthma. “Treatment”, “treating” or “treated” as used herein, means preventing, reducing or eliminating at least one symptom or complication of asthma. Exemplary symptoms and/or complications of asthma include, but are not limited to, AHR, mucus hyperproduction, elevated serum IgE levels, elevated airway eosinophilia and airway remodeling. These methods include administering to a mammal, e.g., a human, in need thereof a therapeutic amount of an agent that decreases the production or activity of a Gob-4 protein. A “therapeutic amount” represents an amount of an agent that is capable of inhibiting or decreasing the production of Gob-4 protein and causes a clinically significant response. The clinical response includes an improvement in the condition treated or in the prevention of the condition. The particular dose of the agent administered according to this invention will, of course, be determined by the particular circumstances surrounding the case, including the agent administered, the particular asthma being treated and similar conditions.

Agents that decrease the activity of a Gob-4 protein include those agents discovered in the screening assays described herein. Additional agents, or inhibitors, are well known in the art and include, for example, IL-13Rα2 and antibodies against a Gob-4 protein. See, e.g., Houston, et al., PNAS 99(14): 9127-9132 (2002). An antibody against the Gob-4 protein as used herein may be, without limitation, a polyclonal antibody, a monoclonal antibody, a chimeric antibody, a humanized antibody, a genetically engineered antibody, a bispecific antibody, antibody fragments (including but not limited to “Fv,” “F(ab′)2,” “F(ab),” and “Dab”) and single chains representing the reactive portion of the antibody. Methods for production of each of the above antibody forms are well known to the art.

In another aspect, the invention provides methods for treating asthma that includes administering to a mammal in need thereof a therapeutic amount of an agent that decreases the production of a Gob-4 protein. In one embodiment, the mammal is a human. In another embodiment, the agent is administered in a pharmaceutically acceptable carrier.

In one embodiment, the agent that decreases the production of the Gob-4 protein is a nucleic acid. Exemplary nucleic acids include, but are not limited to, a deoxyribonucleic acid or a ribonucleic acid. In one embodiment, the ribonucleic acid has a nucleotide sequence that is complementary to a portion of the nucleotide sequence set forth in SEQ ID NO:1, SEQ ID NO:3 or SEQ ID NO:5, as set forth in FIGS. 1, 3 and 5, encoding acidic mammalian protein.

In another embodiment, RNA interference may be used as an inhibitor of Gob-4 protein. RNA interference relates to sequence-specific, posttranscriptional gene silencing brought about by double-stranded RNA that is homologous to the silenced gene target. Methods for inhibiting production of a protein utilizing small interfering RNAs are well known to the art, and disclosed in, for example, PCT International Application Numbers WO 01/75164; WO 00/63364; WO 01/92513; WO 00/44895; and WO 99/32619.

The agents may be administered by a wide variety of routes. Exemplary routes of administration include oral, parenteral, transdermal, and pulmonary administration. For example, the agents may be administered intranasally, intramuscularly, subcutaneously, intraperitonealy, intravaginally and any combination thereof. For pulmonary administration nebulizers, inhalers or aerosol dispensers may be used to deliver the therapeutic agent in an appropriate formulation (i.e., with an aerolizing agent). In addition, the agents may be administered alone or in combination with other agents or known drugs. In combination, agents may be administered simultaneously or each agent may be administered at different times. When combined with one or more known asthma drugs, agents and drugs may be administered simultaneously or the agent can be administered before or after the drug(s).

In one embodiment, the agents are administered in a pharmaceutically acceptable carrier. Any suitable carrier known in the art may be used. Carriers that efficiently solubilize the agents are preferred. Carriers include, but are not limited to a solid, liquid or a mixture of a solid and a liquid. The carriers may take the form of capsules, tablets, pills, powders, lozenges, suspensions, emulsions or syrups. The carriers may include substances that act as flavoring agents, lubricants, solubilizers, suspending agents, binders, stabilizers, tablet disintegrating agents and encapsulating materials.

Tablets for systemic oral administration may include excipients, as known in the art, such as calcium carbonate, sodium carbonate, sugars (e.g., lactose, sucrose, mannitol, sorbitol), celluloses (e.g., methyl cellulose, sodium carboxymethyl cellulose), gums (e.g., arabic, tragacanth), together with disintegrating agents, such as maize, starch or alginic acid, binding agents, such as gelatin, collagen or acacia and lubricating agents, such as magnesium stearate, stearic acid or talc.

In powders, the carrier is a finely divided solid, which is mixed with an effective amount of a finely divided agent.

In solutions, suspensions or syrups, an effective amount of the agent is dissolved or suspended in a carrier such as sterile water or an organic solvent, such as aqueous propylene glycol. Other compositions can be made by dispersing the inhibitor in an aqueous starch or sodium carboxymethyl cellulose solution or a suitable oil known to the art.

The agents are administered in a therapeutic amount. Such an amount is effective in treating asthma. This amount may vary, depending on the activity of the agent utilized, the nature of the asthma and the health of the patient. The term “therapeutically effective amount” is used to denote treatments at dosages effective to achieve the therapeutic result sought. Furthermore, a skilled practitioner will appreciate that the therapeutically effective amount of the agent may be lowered or increased by fine tuning and/or by administering more than one agent, or by administering an agent with an anti-asthmatic compound (e.g., corticosteroid). As illustrated in the following examples, therapeutically effective amounts may be easily determined, for example, empirically by starting at relatively low amounts and by step-wise increments with concurrent evaluation of beneficial effect. (i.e., reduction in asthmatic symptoms following exposure to antigen).

When the agents are combined with a carrier, they may be present in an amount of about 1 weight percent to about 99 weight percent, the remainder being composed of the pharmaceutically acceptable carrier.

Reference will now be made to specific examples illustrating the invention. It is to be understood that the examples are provided to illustrate preferred embodiments and that no limitation to the scope of the invention is intended thereby.

### EXAMPLE 1

### Gene Expression Changes in Mouse Lung Associated with Allergic Reaction

To identify the gene expression changes induced by intratracheal OVA-challenge Balb/C mice (Jackson Laboratories (Bar Harbor, Me.)) were immunized by an intraperitoneal (i.p.) injection of 10 μg of ovalbumin (OVA) (Sigma, St. Louis, Mo.) in 200 μl of PBS on day 0. On days 14 and 25, mice were anesthetized with a mixture of ketamine and xylazine (45 and 8 mg/kg respectively) and challenged intratracheally with 50 μl of a 1.5% solution of OVA or an equivalent volume of PBS. To identify changes in mRNA concentration dependent on IL-13 mediated signal transduction, two of the OVA-challenged mice were treated with three intraperitoneal injections of the soluble IL-13 receptor fusion protein, sIL-13Rα2-Fc, prior to and during the course of the allergic challenge. As control for the Fc-moiety of the receptor fusion protein, two of the OVA-challenged mice were similarly treated with intraperitoneal administration of hIgG. A second set of six control mice were similarly sensitized to OVA without subsequent challenge and treated on an identical time course with intratracheal administration of PBS buffer, either alone (n=2) or with intraperitoneal injection of hIgG (n=2) or sIL-13Rα2-Fc (n=2). Lung tissue for the OVA-challenged and buffer-alone control mice was harvested at 78 hr following the second pulmonary antigen challenge (day 28).

Snap frozen mouse lung tissue was pulverized using liquid nitrogen chilled mortar and pestle, suspended in 6 ml 4M guanidinium isothiocyanate/0.7% 2-mercaptoethanol (GTC/ME) and pulse sonicated for 2 minutes. The tissue suspension was extracted twice with acid equilibrated phenol (Promega Total RNA Kit) and nucleic acid precipitated with an equal volume of isopropanol. The pellet was resuspended in 0.8 ml GTC/ME, reextracted twice with an equal volume of acid phenol and once with chloroform. RNA was ethanol-precipitated, suspended in DEPC treated H2O and quantified by OD280.

cDNA was synthesized from 10 μg of total RNA using the Superscript Kit (BRL) with modifications described in Byrne, et al., “Preparation of mRNA for expression monitoring,” Current Protocols in Molecular Biology, John Wiley and Sons, Inc. (New York 2000). First strand synthesis was carried out at 50° C. to prevent mispriming from ribosomal RNA and utilized a T7 RNA polymerase promoter containing poly-T primer (T7T24) for subsequent in vitro antisense RNA (cRNA) amplification and biotin labeling. cDNA was purified using BioMag Carboxyterminated beads (Polysciences) according to manufacturer's instructions, and eluted in 48 μl of 10 nM NaAcetate pH 7.8.

In vitro T7 polymerase driven transcription reactions for synthesis and biotin labeling of antisense cRNA, Qiagen RNeasy spin column purification and cRNA fragmentation were carried out. GeneChip® hybridization mixtures contained 10 μg fragmented cRNA, 0.5 mg/ml acetylated BSA, 0.1 mg/ml herring sperm DNA, in 1X MES buffer in a total volume of 200 μl as per manufacturer's instructions. Reaction mixtures were hybridized for 18 hours at 45° C. to Affymetrix Mu11KsubA and Mu11KsubB oligonucleotide arrays. The hybridization mixtures were removed and the arrays were washed and stained with Streptavidin R-phycoerythrin (Molecular Probes) using the GeneChip® Fluidics Station 400 and scanned with a Hewlett Packard GeneArray Scanner following manufacturer's instructions. Fluorescent data was collected and converted to gene specific difference averages using MicroArray Suite 4.0 software.

An eleven-member standard curve was prepared by spiking gene fragments derived from cloned bacterial and bacteriophage sequences into each hybridization mixture at concentrations ranging from 0.5 pM to 150 pM. These standards represented RNA frequencies of approximately 3.3 to 1000 parts per million (ppm) assuming an average transcript size of 2 kb. The biotinylated standard curve fragments were synthesized by T7 polymerase driven IVT reactions from plasmid-based templates. The spiked biotinylated RNA fragments served both as an internal standard to assess chip sensitivity and as standard curve to convert measured fluorescent difference averages from individual genes into RNA frequencies in ppm. Average fluorescence difference between perfect match and single mismatch probe sets containing gene-specific oligonucleotides were used to determine frequency values with respect to spiked standard curve. In addition, a second set of algorithms based primarily on the fraction of individual positive or negative responding probe pairs, was used to assess the absolute presence or absence of the gene product. The sensitivity of the individual microarray chip was set at one-half the minimum concentration at which 2 or any 3 adjacent standard curve spike-in templates were called present. The standard curve linear regression was forced through zero and the minimum reported gene frequency was set to the sensitivity of the individual GeneChip®.

Multiple independent replicas for each of the treatment or control experimental conditions were measured and the expression data subjected to routine statistical analysis in an effort to remove false positives. Frequency values determined from individual measurements for a given experimental set were initially compared. Average values for treatment and control animals were compared to obtain average fold change (AFC). Two tailed Student t-tests were calculated using either unequal covariance with raw frequency values or equal covariance with log-transformed frequency values.

The overall gene expression measured for each of the three treatment groups used to identify allergen-challenge induced gene expression was well balanced with respect to mRNA integrity, number of genes called present and total mRNA frequency computed across the various control and treatment files (data not shown).

The gene expression profile measured for control mice treated with PBS was not significantly altered by intraperitoneal co-administration of human IgG or sIL-13Rα2-Fc, and thus frequency values from the six control mice were combined as a single set in the calculation of average untreated baseline expression values. Similarly, the four OVA-challenged mice treated either with intraperitoneal co-administration of buffer or hIgG were combined as a single set in calculation of average frequency values for pulmonary allergen-challenged mRNA frequency. The data are shown on Table 2 below.

TABLE 2
mRNA frequency values for Gob-4 protein following challenge

| Sample | mRNA Frequency (ppm) |
|---|---|
| PBS Control | 6.5 |
| OVA and sILRα2-Fc | 7.0 |
| OVA | 40.8 |

The data demonstrate that Gob-4 protein mRNA is specifically induced by direct pulmonary intratracheal administration of IL-13 or ovalbumin-induced allergic challenge. Additionally, these data show that inhibition of IL-13 activating using the soluble receptor antagonist (sIL-13Rα2-Fc) completely inhibits the expression of Gob-4 protein by ovalbumin challenge. Physiological studies utilizing the sIL-13Rα2-Fc antagonist have previously shown IL-13 activity is essential to asthma disease pathology, including epithelial mucus production and AHR. Thus, Gob-4 protein is identified as an IL-13 responsive gene downstream of the ovalbumin allergic challenge, which identifies it as a therapeutic agent in the target of asthma.

These data confirm that intraperitoneal administration of the soluble IL-13 antagonist was able to completely inhibit the induction of OVA allergic pulmonary Gob-4 protein. Physiological studies utilizing the sIL-13Rα2-Fc antagonist has previously shown to be IL-13 activity essential for asthma disease pathology including epithelial mucus production and AHR. Wills-Karp, M, et al., Science 282(5397): 2258-61 (1998). Thus, these data demonstrate that therapeutic intervention resulting in the inhibition of Gob-4 protein is associated with a decrease of asthma related symptoms.

### EXAMPLE 2

### Gene Expression Changes in Mouse Lung Induced by mIL-13 Lung Instillation

To identify IL-13 mediated changes in pulmonary gene expression, six Balb/C mice (Jackson Laboratories, Bar Harbor, Me.) were treated with multiple 5 μg dose (0, 24 hr, and 48 hr) lung instillation of recombinant mIL-13. A second set of control Balb/C mice (n=4) were instilled with buffer alone on an identical schedule. Additionally, a set of Stat6−/− null mice were treated identically with multiple doses mIL13 (n=4) or PBS buffer (n=5) lung instillation prior to harvesting of all lungs at 78 hr for expression profiling. Stat6−/− is an additional control; it is a key intermediate in IL-13 signaling pathway, critical for mucus production and AHR; the absence of this IL-13 signaling transducer ameliorates asthmatic symptoms. The overall gene expression for each of the three treatment groups used to identify allergen-challenge induced gene expression was well-balanced with respect to mRNA integrity, number of genes called present, and total mRNA frequency computed across the various control and treatment files. The data are shown in Table 3.

TABLE 3

| | | Gob-4 protein mRNA |
|---|---|---|
| Animal | Treatment | Frequency (ppm) |
| Balb/C | PBS | 9.8 |
| | rIL-13 | 108.8 |
| Stat6^{-/-} | PBS | 7.8 |
| | rIL-13 | 11.8 |

These data confirm that increases in Gob-4 mRNA concentration are mediated by mIL-13 lung instillation.

### EXAMPLE 3

### Prophetic Example of Screening Assay for Modulator of Gob-4 Protein Activity

Human Gob-4 protein (hGob-4 protein) is cloned into bacterial expression vector, transformed into E. coli or COS cells and the protein purified from bacterial or mammalian cultures by column chromatography utilizing standard molecular biology and biochemistry methods. Primary epithelial cells or epithelial cell lines are then treated with the Gob-4 protein. After contact with the protein, the cells or cell lines are assayed for an increase in goblet cells or goblet cell-expressed mucin. Test agents will be screened by their ability to modulate (e.g., inhibit) the mucus production as determined by staining of the epithelial cells or by microscopic observation of increased secretory granuoles.

### EXAMPLE 4

### Prophetic Example of Screening Assay for Inhibitor of Gob-4 Protein Production Involving Gob-4 Protein Promoter

A Gob-4 protein promoter is linked to a reporter gene, for example, a luciferase. Activation of the reporter gene is demonstrated by inducing with IL-13, indicating transcriptional specificity. Test agents are screened to identify those that block the IL-13 inducted reporter gene activity.

### EXAMPLE 5

### Treating Asthma with Gob-4 Protein Inhibitor

A therapeutically effective amount of a known Gob-4 protein inhibitor is administered to a subject diagnosed with asthma. A control group also exhibiting asthmatic symptoms will be a placebo control. Administration may be by a single treatment or treatment over a course of days. Subjects are evaluated for asthma-related symptoms, such as AHR, forced expiratory volume and mucus production, following pulmonary antigen challenge. Effective treatment is determined by a reduction in asthma-related symptoms compared to the control group.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A method of screening for agents for treating asthma in a human, comprising:
(a) contacting a mammalian Gob-4 protein with a test agent thought to be effective in inhibiting the activity of said mammalian Gob-4 protein;
(b) determining if said test agent inhibits the activity of said mammalian Gob4 protein, wherein determining if said test agent inhibits the activity of said mammalian Gob-4 protein comprises quantitating the amount of mucus produced by staining said mucus with a periodic-acid Schiff stain; and
(c) classifying said test agent as an agent for treating asthma if said test agent inhibits the activity of said mammalian Gob4 protein.

2. A method of screening for agents for treating asthma in a mammal, comprising:
(a) contacting a Gob-4 protein with a test agent thought to be effective in inhibiting the activity of said Gob-4 protein;
(b) determine if said test agent inhibits the activity of said Gob-4 protein; and
(c) classifying said test agent as an agent for treating asthma if said test agent inhibits the activity of said Gob-4 protein.

3. The method of claim 2, wherein determining if said test agent inhibits the activity of said mammalian Gob-4 protein comprises determining the number of goblet cells that form from epithelial cells.

4. The method of claim 2, wherein determining if said test agent inhibits the activity of said mammalian Gob-4 protein comprises quantitating the amount of mucus produced.

5. The method of claim 4, wherein said quantitating the amount of mucus produced comprises quantitating the amount of mucopolysaccharides in said mucus.

6. The method of claim 2, wherein said Gob4 protein has an amino acid sequence selected from the group consisting of SEQ ID NO:2 and SEQ ID NO:4.

7. The method of claim 2, wherein said Gob-4 protein has an amino acid sequence having at least about 70% identity to an amino acid sequence selected from the group consisting of SEQ ID NO:2 and SEQ ID NO:4.

8. A method of screening for agents for treating asthma in a mammal, comprising:
(a) contacting a nucleotide sequence encoding a reporter gene product operably linked to a Gob-4 protein promoter with a test agent thought to be effective in inhibiting production of a Gob-4 protein;
(b) determining if said test agent inhibits production of said reporter gene product; and
(c) classifying said test agent as an agent for treating asthma if said test agent inhibits production of said reporter gene product.

9. The method of claim 8, wherein determining if said test agent inhibits production of said Gob-4 protein comprises quantifying the amount or activity of said reporter gene product.

10. The method of claim 8, wherein said Gob-4 protein promoter has a nucleotide sequence selected from the group consisting of SEQ ID NO:5.

11. The method of claim 8, wherein said Gob-4 protein promoter has a nucleotide sequence having at least about 80% identity to the nucleotide sequence selected from the group consisting of SEQ ID NO:5.

12. The method of claim 8, wherein said Gob-4 protein promoter has a nucleotide sequence selected from the group consisting of SEQ ID NO:6.

13. The method of claim 8, wherein said Gob-4 protein promoter has a nucleotide sequence having at least about 80% identity to the nucleotide sequence selected from the group consisting of SEQ ID NO:6.

14. The method of claim 8, wherein said reporter gene product is selected from the group consisting of luciferase, β-galactosidase, chloramphenical acetyltransferase, β-glucuronidase, alkaline phosphatase, and green fluorescent protein.

15. A method for treating asthma, comprising administering to a mammal in need thereof a therapeutic amount of an agent that decreases the activity of a Gob-4 protein.

16. A method for treating asthma, comprising administering to a mammal in need thereof a therapeutic amount of an agent that decreases the production of a Gob-4 protein.

17. The method of claim 16, wherein said agent that decreases the production of said Gob-4 protein is a nucleic acid.

18. The method of claim 17, wherein said nucleic acid is a ribonucleic acid.

19. The method of claim 18, wherein said ribonucleic acid has a nucleotide sequence that is complementary to a portion of the nucleotide sequence set forth in SEQ ID NO:1 or SEQ ID NO:3 encoding said acidic mammalian protein.

20. The method of claim 18, wherein said ribonucleic acid is RNA interference.
